(19) United States
(12) Patent Application Publication
(10) Pub. No.: US 2007/0078116 A1
(43) Pub. Date: Apr. 5, 2007

(54) Method of treatment of otitis externa

(75) Inventor: Edward M. Lane, Weston, CT (US)

Correspondence Address: ROTHWELL, FIGG, ERNST & MANBECK, P.C., 1425 K STREET, N.W., SUITE 800, WASHINGTON, DC, 20005, US

(73) Assignee: Fairfield Clinical Trials, LLC, Bridgeport, CT (US)

(21) Appl. No.: 11/543,203

(22) Filed: Oct. 5, 2006

Related U.S. Application Data
(63) Continuation-in-part of application No. 10/771,330, filed on Feb. 5, 2004.
(60) Provisional application No. 60/496,409, filed on Aug. 20, 2003.
(60) Provisional application No. 60/505,754, filed on Sep. 26, 2003.

(51) Int. Cl. A61K 31/573; A61K 31/496; A61K 31/4164

(52) U.S. Cl. 514/171; 514/396; 514/254.07

(57) ABSTRACT

This invention relates to a method of treating otitis externa, and in particular otitis externa of fungal etiology, using topical medication, including antifungal agents such as itraconazole and miconazole, and optionally also including a second antifungal agent, which is formulated for topical application to the external ear canal, preferably as an ear drop in liquid form.

This application is a continuation-in-part of copending application Ser. No. 10/771,330 filed Feb. 5, 2004, which claims benefit of prior co-pending U.S. Provisional Application Ser. No. 60/496,409, filed Aug. 20, 2003, and co-pending U.S. Provisional Application Ser. No. 60/505,754, filed Sep. 26, 2003, the disclosures of all of which are hereby incorporated by reference.

### BACKGROUND OF THE INVENTION

1. Technical Field

This invention relates to the field of medical science, and in particular to treatment of otitis externa, and particularly otitis externa of fungal etiology, with topical or orally administered antifungal agents, preferably including fluconazole, voriconazole, itraconazole, clotrimazole, miconazole and amphotericin B.

2. Description of the Background Art

The external auditory canal is the longest skin-lined cavity in the body. Otitis externa is an inflammation of the external auditory canal which can affect people of all ages. This condition is responsible for considerable pain and morbidity. The cause may be bacterial (usually Staphylococcus spp.), viral (for example herpes zoster oticus), traumatic (usually caused by aggressive ear cleaning), collection (or appearance) of moisture or water under a cerumen impaction and/or fungal. Statistically, in the United States, 1 in 4000 patients will get otitis externa annually. The incidence for fungal otitis externa is roughly 10% of these.

Otitis externa infections often involve a mixed population of bacteria and fungus. Fungal otitis externa (otomycosis) is a fungal infection of the external auditory canal and generally is caused by (1) Aspergillus niger or A. fumigatus (80-90% of all cases), (2) Candida albicans and other Candida spp., (3) Actinomyces and (4) Trichophyton. Factors such as hot, humid environments, chronic bacterial otitis externa, prior treatment of bacterial otitis externa with topical aminoglycosides or other antibacteriologics and suppressed immunity can predispose patients to fungal otitis externa. The number of persons at risk for this infection is increasing due to the liberal and inappropriate use of systemic antibiotics, the increase in patients undergoing bone marrow transplant, solid organ transplant, aggressive chemotherapy for cancer and patients infected with HIV.

Symptoms of otitis externa can include significant ear canal pruritis, pain (particularly with motion of the external ear), otorrhea (usually foul and purulent), conductive hearing loss and cervical lymphadenitis. Whitish-grey, yellow or black ear canal exudate, erythema and swelling of the canal walls, external auditory canal meatus and tympanic membrane, and a distinctive odor are hallmarks of the condition. Other symptoms may include hearing loss, tinnitus, fever and others. If the infection is severe, it may spread through the skin layers to cartilage and/or bone, and can spread to the face or neck. Necrotizing or malignant otitis externa, a Pseudomonas spp. ostiitis of the temporal bone, may occur, especially in adults with diabetes mellitus, both Types I and II, as well as in patients who are immunocompromised. Diagnosis of otitis externa often is confirmed by staining a sample of the exudate with potassium hydroxide (10% KOH) or fungal culture, although most patients are diagnosed empirically.

Treatment of otitis externa involving fungal organisms generally entails vigorous ear canal cleaning (ear toilet), irrigation and acidification. Occasionally, surgical debridement of the ear canal is indicated. Current therapy for fungal otitis externa relies on the use of acidifying solutions (for example acetic acid, with or without hydrocortisone) or topical agents designed for treatment of Athlete's Foot (for example clotrimazole Lotrimin®). Acidifying agents generally are ineffective against Aspergillus species, however. Antifungal topical agents such as Lotrimin® are designed for treatment of candidiasis, but generally are not efficacious for many of the organisms known to cause fungal otitis externa and so have proved ineffective. Topical antibiotic preparations have been in use for many years to treat otitis of bacterial origin. There are, however, no topical or systemic medications indicated for treatment or prophylaxis of fungal otitis externa that contain effective antifungal compounds commercially available at this time.

Orally active antifungal drugs have been described. See U.S. Pat. No. 4,404,216. These drugs have been used effectively for invasive fungal infections due to Candida, Aspergillus, and other fungi. Azole antifungal agents such as fluconazole and voriconazole exert their effect by inhibiting cytochrome p450 14a-desmethylase (P45014DM), an enzyme in the steroid biosynthesis pathway. Voriconazole has in vitro antifungal activity against a number of species and is considered to be effective in vivo against Candida spp. and Cryptococcus neoformans as well as Aspergillus spp., including fluconazole-resistant Candida species such as C. krusei and C. guilliermondii. Miconazole (Monistat®) is commercialized for Candida vaginal infections. Fluconazole (Diflucan®), itraconazole (Sporanox®), voriconazole (Vfend®) and clotrimazole (Mycelex®) have been approved by the FDA for various types of invasive fungal infections. These drugs are synthetic triazole antifungal agents, available as tablets for oral administration. Prescribing information for these drugs list the following indications for usage. Fluconazole: vaginal candidiasis; oropharyngeal and esophageal candidiasis; Candida urinary tract infections, peritonitis, and systemic Candida infections including candidemia, disseminated candidiasis, and pneumonia; and cyptococcal meningitis. Voriconazole: invasive aspergillosis and serious fungal infections caused by Scedosporium apiospermum and Fusarium spp. Itraconazole: blastomycosis, histoplasmosis and aspergillosis in immunocompromised patients and onychomycosis in non-immunocompromised patients. Fluconazole also has been used to decrease the incidence of candidiasis in patients undergoing bone marrow transplantation who receive cytotoxic chemotherapy and/or radiation therapy.

### SUMMARY OF THE INVENTION

An objective of certain embodiments of this invention is to provide a treatment for fungal otitis externa in a patient, using topical antifungal medication.

Accordingly, embodiments of this invention provide a method of treating otitis externa in a patient in need thereof, which comprises topically administering to said patient a therapeutically effective amount of an antifungal agent such as miconazole. Other preferred antifungal agents are fluconazole, voriconazole, itraconazole, clotrimazole and amphotericin B. Other suitable antifungal agents include, but are not limited to caspofungin (Cancidas®), micafungin (Mycamine®), terbinafine, naftifine, natamycin, butenafine, amorolfine, ravuconazole, posaconazole, flucytosine, econazole, enilaconazole, miconazole, oxiconazole, saperconazole, sulconazole, terconazole, tioconazole, nikkomycin Z, anidulafungin (LY303366), nystatin, pimaricin, griseofulvin, ciclopirox, haloprogin, tolnaftate, and undecylenate. For topical treatment, the agent is administered in an amount of about 0.001 mg/day to about 5,000 mg/day, preferably 1 mg/day to about 1,000 mg/day or about 5 mg/day to about 500 mg/day and most preferably about 10 mg/day to about 100 mg/day. The drug concentration applied may be about 0.05% to about 10%, about 0.1% to about 5%, or about 0.3% to about 3.0%, each by weight of the total composition. Treatment preferably should be administered at least once daily and preferably twice daily to four (4) times daily for one day or at least 3 days or 10 days, preferably for about 7 days to about 14 days. Treatment can be for 180 days or longer. The methods are suitable for treating otitis externa that is non-invasive or invasive. Preferred methods are suitable for treating otitis externa in which a Candida or Aspergillis species such as C. albicans, A. niger and/or A. Fumigatus is a causative agent.

### DESCRIPTION OF THE FIGURES

FIG. 1 is a photograph showing drug effects on C. albicans.

FIG. 2 is a photograph showing drug effects on A. fumigatus.

FIG. 3 is a photograph showing drug formulation effects on C. Albicans.

FIG. 4 is a photograph showing drug formulation effects on A. Fumigatus.

A number of preferred aspects of the invention will be described below.

### DETAILED DESCRIPTION OF THE INVENTION

Antifungal agents preferably are delivered to the affected tissue as a liquid, such as in a solution or suspension, by medicine dropper. Such solutions or suspensions generally contain about 0.001 mg to about 5000 mg antifungal agent per mL of solution or suspension in a suitable pharmaceutical carrier, but may contain about 1 mg to about 2,500 mg agent per mL solution or suspension, and preferably about 5 mg to about 1,500 mg agent per mL solution or suspension or about 10 mg to about 1,000 mg agent per mL solution or suspension. The solution or suspension can be delivered to the ear canal in amounts of about 0.01 mL to about 5 mL, preferably about 0.1 mL to about 1 mL, or any amount sufficient to fill the canal volume. An ear wick may be used to assist penetration of the agent into the ear canal according to methods known in the art.

Typical treatments with topical formulations involve administration of a 1% solution or suspension of the antifungal agent as described herein, twice daily to four (4) times daily or at least once daily for 10 days. The length of treatment preferably is at least 3 days or 10 days, but may extend from 1 day to about 14 days, or until the symptoms are resolved. Preferably, treatment continues for 5 days after resolution of symptoms to lessen chance of recurrence. Treatment may be given for extended periods, for example up to 180 days or more, if necessary. Solutions/suspensions at concentrations of about 0.1% to 5% or about 0.3% to 2% or any concentration in these ranges also may be used and are contemplated for use with the invention.

Solutions and suspensions of these types as liquid formulations are known in the art and may contain any conventional or pharmaceutically acceptable and suitable excipients. Alternatively, the azole or other antifungal agents may be formulated as an ointment, lotion, cream, tincture, paste, aqueous or anhydrous gel, or powder according to traditional methods known in the pharmaceutical arts and using any conventional and acceptable pharmaceutical excipient or excipients that are known in the art. A preferred excipient or vehicle for formulations according to certain embodiments of the invention is a 2% glacial acetic acid, 1% hydrocortisone solution in propylene glycol, optionally also containing a preservative such as benzalkonium chloride or benzethonium chloride.

Inert ingredients or other excipients which advantageously may be used in embodiments of the invention include preservatives such as benzalkonium or benzethonium compounds, or any preservative suitable for the formulation. Such preservatives for liquid pharmaceutical formulations are well known in the art and any of these are contemplated for use with embodiments of the invention. In addition, any conventional solvent, buffer or pH adjustment agent, colorant, viscosity enhancer, surfactant or wetting agent, solubility enhancer or any other pharmaceutically acceptable excipient or vehicle is appropriate for use with embodiments of the invention.

Topical preparations for application to the external ear canal according to the invention generally are formulated as a liquid and are applied as ear drops, for example using about 4 drops, to the affected ear canal of a patient with eardrum held dependently. Other methods for administration of other types of topical formulations are known in the art, however, and these also are contemplated for use with the invention.

Formulations of azole antifungal agents suitable for use with this invention may contain additional ingredients which may have activity against microorganisms or which may assist in alleviating symptoms in addition to inert pharmaceutical excipients. For example, topical formulations may include hydrocortisone or other corticosteroid agents to assist in reducing inflammation. Such corticosteroids (for example hydrocortisone or dexamethasone) are able to provide synergistically improved effects in topical formulations. Formulations may contain acidifying agents such as acetic acid, anesthetic agents such as pramoxine HCl, lidocaine or pontocaine, solvents such as propylene glycol diacetate, or antibacterial agents such as telithromycin, if desired.

Formulations according to the invention preferably contain miconazole, itraconazole or voriconazole, which are effective against Aspergillus spp. and Candida spp., common causes of fungal otitis externa. Other agents which may form part of the invention include fluconazole, ketoconazole, enilaconazole, econazole, saperconazole, oxiconazole, clotrimazole, amphotericin B, caspofungin, micafungin, terbinafine, naftifine, natamycin, butenafine, amorolfine, ravuconazole, posaconazole, flucytosine, sulconazole, terconazole, tioconazole, nikkomycin Z, anidulafungin (LY303366), nystatin, pimaricin, griseofulvin, ciclopirox, haloprogin, tolnaftate, and undecylenate.

A preferred composition of the invention contains 0.3-3% itraconazole or miconazole; 2% glacial acetic acid; 1% hydrocortisone; 0.02% benzethonium chloride (optional); 0.015% sodium acetate trihydrate (optional) and 0.2% citric acid (optional) in propylene glycol. A second preferred composition of the invention contains: 0.3-3% itraconazole or miconazole; 10 mg/mL hydrocortizone; 10 mg/mL pramoxine HCl; 1 mg/mL benzalkonium chloride; 3% propylene glycol diacetate in a non-aqueous propylene glycol solvent system. Any excipient such as solvents, buffers, preservatives, colorants, scents, fillers, surfactants, wetting agents, or any pharmaceutically acceptable excipient also may be used.

Treatment methods of the invention for treatment of otitis externa (infections of the external ear canal) may include any antifungal agent which is effective for the particular causative species of fungus. When the specific causative fungus is not or cannot be identified, or when more than one fungus is present or suspected, miconazole, itraconazole, voriconazole or a combination thereof preferably is used, alone or in combination with other active agents.

Topical medications such as powders and creams which are designed to treat athlete's foot sometimes have been used in the ear to treat otitis of fungal origin, however these products, containing clotrimazole or fluconazole for example, do not effectively treat most otitis externa. Agents designed to treat athlete's foot may be effective against some Candida species, but are not suitable alone in a general formulation for otitis externa because in most cases, the causative agent(s) are not known and may not be Candida species. Therefore, these pharmaceutical compositions, which are not effective against Aspergillus niger and other Aspergillis species, the most common causative organism, preferably are not used alone but may be used as an additional active ingredient in the inventive compositions. Miconazole and itraconazole both were highly active in killing fungi of both Aspergillus and Candida species in vitro and the clinical results from studies involving patients suffering from otitis-externa of fungal origin show the effectiveness of itraconazole. See Examples.

Preferred topical preparations contain one or more additional antifungal compounds such as those listed above and most preferably contain miconazole, itraconazole and/or voriconazole. In addition, compounds such as amphotericin B or natamycin are suitable for use as the only antifungal agent. The primary active ingredient, for example miconazole, itraconazole or natamycin, may be combined with a second antifungal agent, an antibacterial agent, an anesthetic, an acidifying agent or buffer, a penetration enhancing agent, an antiinflammatory agent, or a combination of any thereof in a formulation suitable for topical application to the site of infection. Such compositions are effective in the treatment of otitis externa, filling a need in the market, since no effective product is available commercially at this time.

### EXAMPLES

### Example 1.

### Activity of Antifungal Drugs Against Candida and Aspergillis Species.

Candida albicans (ATCC 90028) and Aspergillis fumigatus (ATCC 16424) were tested in vitro for the effect of antifungal compositions according to embodiments of the invention. Fungal cells were obtained from the American Type Culture Collection.

C. Albicans yeast cells were grown in YM broth (Difco™ 0711-01) containing the following ingredients: 1% peptone, 1% yeast extract and 2% glucose. The cells were grown at 37° C., harvested by centrifugation, resuspended in the same medium and stored frozen at −80° C. The cells were used at a concentration of about 1×10⁶ cells/mL. A. fumigatus was cultured on solid MPG containing 20 g/L malt extract (Difco™ 21860); 1 g/L Difco™ 211677); 20 g/L sucrose and 15 g/L Bacto-agar (Difco™ 140-07-04) in water, at 37° C. for 7 to 10 days. Conidia were harvested by flooding the medium with sterile water, filtered through glass wool to exclude hyphal fragments, washed by centrifugation (5000 xg for 10 minutes at 4° C., resuspended at about 1×10⁶ conidia/mL in water and stored at −80° C. until used.

For testing, A. fumigatus conidia and C. albicans cells were thawed. Each cell type was added separately to molten (about 47° C.) YM broth containing 1.5% (w/v) agar at a final concentration of about 1×10³ C. albicans yeast cells or A. fumigatus conidia per mL. The suspensions were mixed well, poured into 150 mm diameter Petri dishes, and the medium allowed to solidify at room temperature.

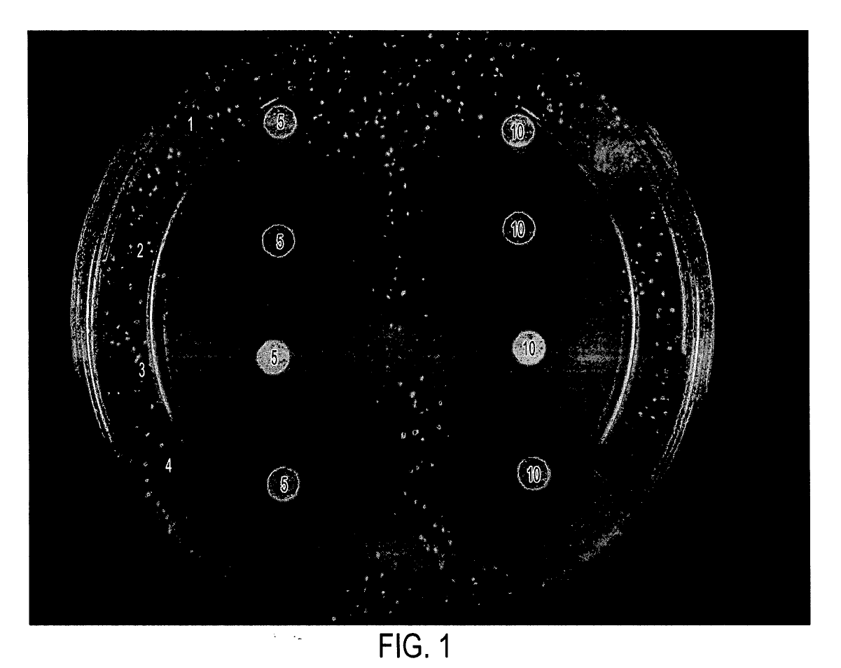

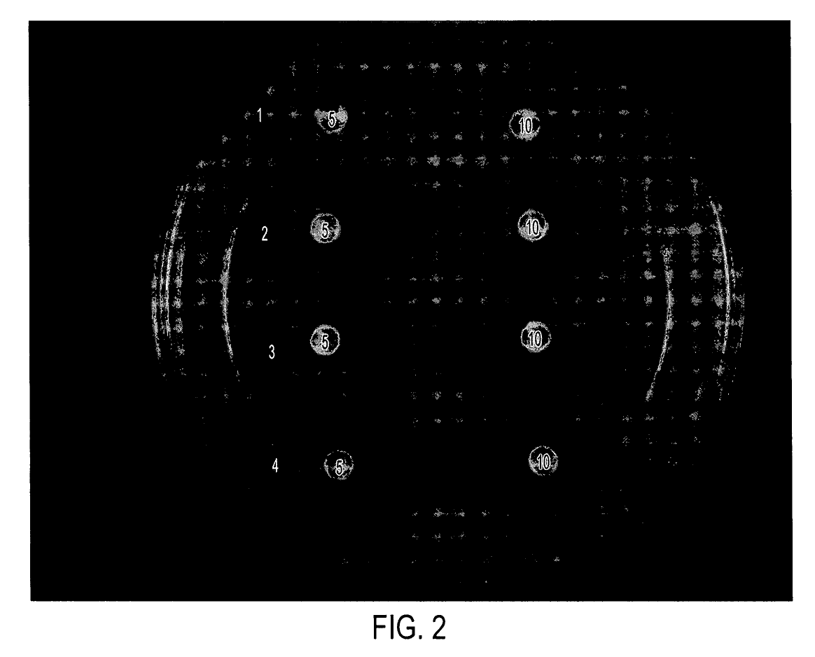

Samples (5 μL, 10 μL or 20 μL as indicated in the Figures) of liquid drug formulation were placed on sterile filter paper disks and the disks allowed to air dry 15-30 minutes at room temperature in a Class II biosafety hood. The disks then were placed gently on the surface of the agar plates containing fungus and the plates incubated at 37° C. for 24-72 hours. The plates were visually observed and photographed using an Alpha Innotech™ digital imaging system. FIG. 1 shows results for Samples 1-4 tested for activity against C. albicans and FIG. 2 against A. fumigatus, using 5 μL and 10 μL of the indicated drug solution as indicated. The samples of drug formulation tested are shown in Table I, below. A portion of each sample was analyzed for potency.

The results show that the miconazole formulations containing 0.3%, 1% and 3% all were effective at killing both C. albicans and A. fumigatus. See FIGS. 1 and 2. The vehicle alone, sample 1, did not exhibit any meaningful activity against either fungus.

TABLE I
Antifungal Drug Formulations.

| | Active | | | |
|---|---|---|---|---|
| Sample | Antifungal | Concentration | Other | |
| No. | Drug | (wt %) | Ingredients | Vehicle |
| 1 | none | 0.0 | 2% glacial acetic acid | 0.02% benzalkonium chloride |
| | | | 1% hydrocortisone | 0.015% sodium acetate trihydrate |
| | | | | 0.2% citric acid in propylene glycol |
| 2 | miconazole | 0.3 | 2% glacial acetic acid | 0.02% benzalkonium chloride |
| | | | 1% hydrocortisone | 0.015% sodium acetate trihydrate |
| | | | | 0.2% citric acid in propylene glycol |
| 3 | miconazole | 1.0 | 2% glacial acetic acid | 0.02% benzalkonium chloride |
| | | | 1% hydrocortisone | 0.015% sodium acetate trihydrate |
| | | | | 0.2% citric acid in propylene glycol |
| 4 | miconazole | 3.0 | 2% glacial acetic acid | 0.02% benzalkonium chloride |
| | | | 1% hydrocortisone | 0.015% sodium acetate trihydrate |
| | | | | 0.2% citric acid in propylene glycol |

### Example 2

### Antifungal Drug Formulations

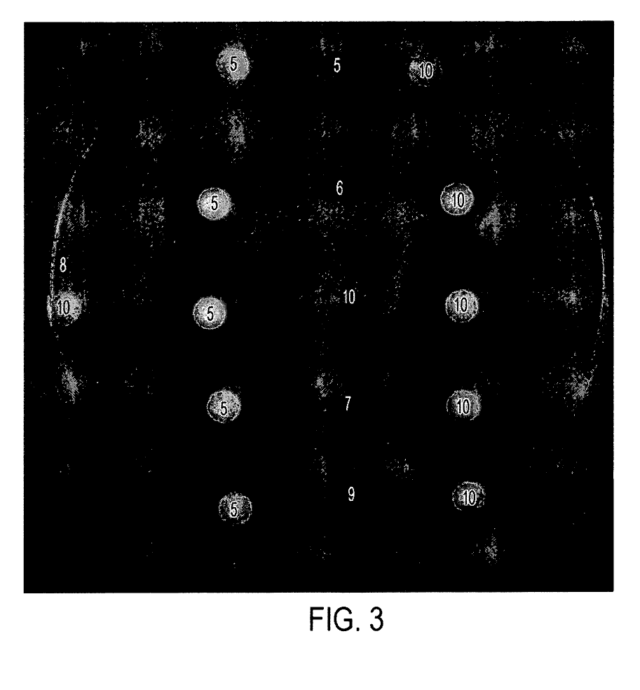

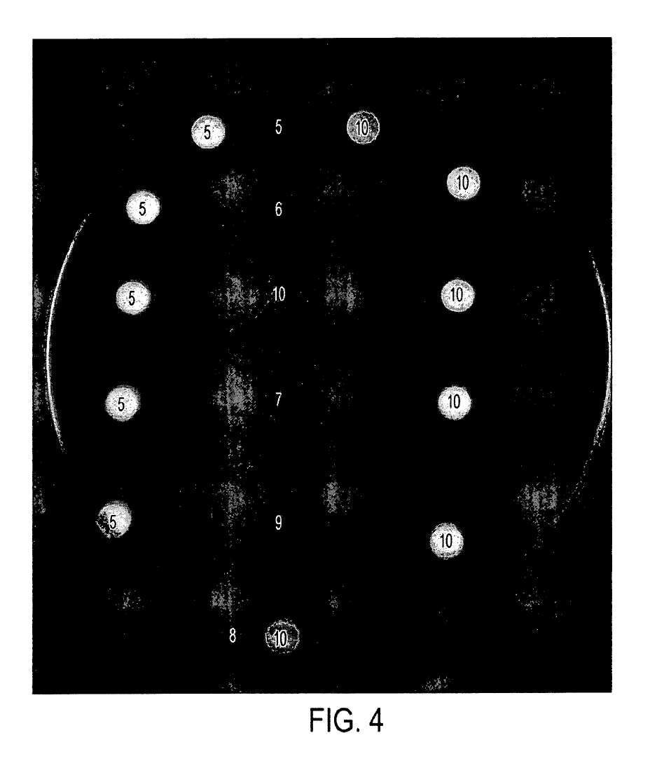

The formulations in Table II below were tested in vitro in the same manner as disclosed in Example 1. Results are shown in FIGS. 3 and 4, and in Table III, below.

Briefly, each of the compositions indicated (5 μL or 10 μl was placed on a sterile filter paper disk. The disks were allowed to air dry and then placed gently on the surface of agar plates containing A. fumigatus or C. albicans or described in Example 1. The plates were incubated for 24-72 hours and photographed. The diameter of the zone of killing for A. fumigatus is provided in Table III.

TABLE II
Drug Formulations.

| Sample | | |
|---|---|---|
| No. | Name | Composition |
| 5 | AA | 2% acetic acid, |
| | | 5% glycerine in isopropanol |
| 6 | AA/I 1% | 2% acetic acid, 1% Itraconazole |
| | | 5% glycerine in isopropanol |
| 7 | AA/M 1% | 2% acetic acid, 1% Miconazole, |
| | | 5% glycerine in isopropanol |
| 8 | G | 5% glycerine in isopropanol |
| 9 | G/I 1% | 1% Itraconazole, |
| | | 5% glycerine in isopropanol |
| 10 | G/M 1% | 1% Miconazole, |
| | | 5% glycerine in isopropanol |

TABLE III
Zone of Killing For Drug Formulations (A. Fumigatus).

| Sample | | Volume | |
|---|---|---|---|
| No. | Name | (μL) | Zone of Killing (mm) |
| 5 | AA | 5 | 10 |
| 6 | AA/I | 5 | 16 |
| 7 | AA/M | 5 | 26 |
| 8 | G | 5 | 0 |
| 9 | G/I | 5 | 21 |
| 10 | G/M | 5 | 22 |
| 5 | AA | 10 | 11 |
| 6 | AA/I | 10 | 20 |
| 7 | AA/M | 10 | 36 |
| 8 | G | 10 | 0 |
| 9 | G/I | 10 | 23 |
| 10 | G/M | 10 | 29 |

FIG. 3 shows data for the indicated samples on C. albicans. The disks are labeled to show the amount of liquid formulation applied to the paper disks (5 μL or 10 μL). FIG. 4 shows the same data for A. fumigatus. The control samples (5 and 8 ) had no significant effect on either fungus, although the 2% aceticc acid solution had a slight effect on A. fumigatus. All formulations containing miconazole or itraconazole exhibited significant killing of both fungi.

### Example 3

### Clinical Testing in Human Patients

A 1% itraconazole solution in a vehicle containing 2% glacial acetic acid, 1% hydrocortisone, 0.02% benzalkonium chloride, 0.015% sodium acetate trihydrate and 0.2% citric acid in propylene glycol or vehicle alone was administered (four (4) drops in the affected ear twice daily for 10 days) to patients diagnosed with otitis externa. See Table IV, below. Six out of the seven patients receiving the 1% itraconazole solution were cured, with the ear canal drying up within 3-5 days; symptom relief generally was achieved by 3-5 days with complete remission of disease seen at 12-15 days (cure). These patients remained disease free at 18-21 days. Vehicle alone was ineffective at curing fungal otitis externa in all but one case. Fungus cultured from the patients was identified and is shown in Table IV, below. Those patients from whom no fungus could be cultured were dropped from the study.

TABLE IV
Clinical Study Outcomes.

| Patient | Patient | Patient | Patient | Composition | | |
|---|---|---|---|---|---|---|
| Number | Name | Age | Gender | Administered | Result | Fungal Etiology |
| 1 | RV | 87 | M | Itraconazole | Cure | A. niger |
| 2 | SL | 65 | F | Vehicle | Fail | C. albicans |
| 3 | CB | 61 | F | Vehicle | Fail | C. parapsilosis |
| 4 | PH | 43 | F | Itraconazole | Cure | A. niger |
| 5 | AP | 38 | M | Itraconazole | Cure | A. flavus |
| 6 | JV | 66 | M | NA¹ | Dropped | NG² |
| 7 | BF | 47 | F | Itraconazole | Cure | C. albicans |
| 8 | DM | 50 | F | NA | Dropped | NG |
| 9 | JB | 48 | F | NA | Dropped | NG |
| 11 | JM | 70 | M | NA | Dropped | NG |
| 12 | WV | 65 | M | Vehicle | Fail | A. niger |
| 13 | VS | 45 | M | NA | Dropped | NG |
| 14 | AC | 82 | M | Itraconazole | Cure | C. albicans |
| 15 | JC | 66 | M | Vehicle | Fail | C. parapsilosis |
| 16 | KC | 18 | F | Vehicle | Cure | C. parapsilosis |
| 17 | BM | 71 | M | NA | Dropped | NG |
| 18 | AL | 43 | F | Vehicle | Fail | C. parapsilosis |
| 19 | RB | 72 | F | Vehicle | Fail | C. parapsilosis |
| 21 | WB | 31 | F | Itraconazole | Cure | C. parapsilosis |
| 22 | AL | 43 | F | Vehicle | Fail | C. parapsilosis |
| 23 | KW | 41 | M | Vehicle | Fail | A. niger |
| 24 | TL | 78 | M | NA | Dropped | NG |
| 26 | NM | 65 | M | Itraconazole | Fail | A. niger |
| 27 | DM | 49 | F | NA | Dropped | NG |
| 28 | AG | 38 | M | NA | Dropped | NG |
| 29 | PV | 46 | F | Vehicle | Fail | A. niger |

¹NA = not applicable
²NG = no growth

1. A method of treating otitis externa in a human patient in need thereof, wherein said otitis externa is caused by Aspergillus species, which comprises topically administering to the Aspergillus-affected external ear canal of said human patient a therapeutically effective amount of a liquid ear drop composition which comprises a miconazole, a corticosteroid anti-inflammatory agent and an acidifying agent.

2. A method of treating otitis externa in a human patient in need thereof, wherein said otitis externa is caused by Aspergillus species, which comprises topically administering to the Aspergillus-affected external ear canal of said human patient a therapeutically effective amount of a liquid ear drop composition which comprises itraconazole, a corticosteroid anti-inflammatory agent and an acidifying agent.

3. A method of treating otitis externa in a human patient in need thereof, wherein said otitis externa is caused by Candida species which comprises topically administering to the Candida-affected external ear canal of said human patient a therapeutically effective amount of a liquid ear drop composition which comprises miconazole, a corticosteroid anti-inflammatory agent and an acidifying agent.

4. A method of treating otitis externa in a human patient in need thereof, wherein said otitis externa is caused by Candida species which comprises topically administering to the Candida-affected external ear canal of said human patient a therapeutically effective amount of a liquid ear drop composition which comprises itraconazole, an anti-inflammatory agent and a corticosteroid acidifying agent.

5. A method of treating otitis externa in a human patient in need thereof, which comprises topically administering to the affected external ear canal of said patient a therapeutically effective amount of a composition which comprises an antifungal agent and at least one pharmaceutically acceptable excipient.

6. A method of treating otitis externa of claim 5 wherein said antifungal agent is miconazole.

7. A method of treating otitis externa of claim 5 wherein said antifungal agent is itraconazole.

8. A method of treating otitis externa of claim 5 wherein said antifungal agent is voriconazole.

9. A method of treating otitis externa of claim 5 wherein said antifungal agent is selected from the group consisting of voriconazole, fluconazole, itraconazole, clotrimazole, ravuconazole, posaconazole, oxiconazole, saperconazole, sulconazole, terconazole, tioconazole, econazole, enilaconazole, miconazole, amphotericin B, natamycin, nikkomycin Z, caspofungin, micafungin, anidulafungin, terbinafine, naftifine, butenafine, amorolfine, flucytosine, nystatin, pimaricin, grisefulvin, ciclopirox, haloprogin, tolnaftate, and undecylate.

10. A method of claim 5 which comprises topically administering to the affected external ear canal of said patient about 1 to about 5,000 mg/day of said antifungal agent.

11. A method of claim 5 which comprises topically administering to the affected external ear canal of said patient about 5 to about 500 mg/day of said antifungal agent.

12. A method of claim 5 which comprises topically administering to the affected external ear canal of said patient 10 mg/day to about 100 mg/day of said antifungal agent.

13. A method of claim 5 wherein said composition comprises 0.3% by weight of said antifungal agent.

14. A method of claim 5 wherein said composition comprises 1.0% by weight of said antifungal agent.

15. A method of claim 5 wherein said composition comprises 3.0% by weight of said antifungal agent.

16. A method of claim 5 wherein said composition comprises about 0.05% to about 10% by weight miconazole, about 0.5% to about 3% hydrocortisone, about 1% to about 4% acetic acid and a pharmaceutically acceptable liquid vehicle.

17. A method of claim 5 wherein said composition comprises about 0.05% to about 10% by weight itraconazole, about 0.5% to about 3% hydrocortisone, about 1% to about 4% acetic acid and a pharmaceutically acceptable liquid vehicle.

18. A method of claim 5 wherein said composition comprises about 0.3% to about 3% miconazole, about 1% hydrocortizone, about 2% acetic acid and a pharmaceutically acceptable liquid vehicle.

19. A method of claim 5 wherein said composition comprises about 0.3% to about 3% itraconazole, about 1% hydrocortizone, 2% acetic acid and a pharmaceutically acceptable liquid vehicle.

20. A method of claim 5 wherein said antifungal agent is administered for at least 3 days.

21. A method of claim 5 wherein said antifungal agent is administered for about 7 days to about 14 days.

22. A method of claim 5 wherein said antifungal agent is administered for up to 180 days.

23. A method of claim 5 wherein said composition further comprises a second agent selected from the group consisting of voriconazole, fluconazole, itraconazole, clotrimazole, miconazole, ravuconazole, posaconazole, oxiconazole, saperconazole, sulconazole, terconazole, tioconazole, econazole, enilaconazole, amphotericin B, natamycin, nikkomycin Z, caspofungin, micafungin, anidulafungin, terbinafine, naftifine, butenafine, amorolfine, flucytosine, nystatin, pimaricin, griseofulvin, ciclopirox, haloprogin, tolnaftate, and undecylenate.

24. A method of claim 5 wherein said composition further comprises an anesthetic agent.

25. A method of claim 5 wherein said composition further comprises an antiinflammatory agent.

26. A method of claim 25 wherein said antiinflammatory agent is a corticosteroid.

27. A method of claim 26 wherein said corticosteroid is hydrocortisone.

28. A method of claim 5 wherein said composition further comprises an acidifying agent.

29. A method of claim 28 wherein said acidifying agent is acetic acid.

30. A method of claim 5 wherein said composition is formulated as a liquid.

31. A method of claim 5 wherein said composition is administered as an ear drop.

32. A method of claim 5 wherein a Candida species is causative organism of said otitis externa.

33. A method of claim 32 wherein said Candida species is C. albicans.

34. A method of claim 5 wherein an Aspergillis species is a causative organism of said otitis externa.

35. A method of claim 34 wherein said Aspergillis species is A. niger or A. fumigatus.
